(19) United States
(12) Patent Application Publication
(10) Pub. No.: US 2018/0353076 A1
(43) Pub. Date: Dec. 13, 2018

(54) METHOD FOR EVALUATING OPERATIONAL PERFORMANCE OF A BIOLOGICAL SYSTEM AND APPARATUS FOR FETCHING DATA REQUIRED THEREFOR

(71) Applicant: PROHEALTH BIOSCIENCE CO., LTD., Taipei (TW)

(72) Inventor: Cheng Hung TAI, Taipei (TW)

(73) Assignee: PROHEALTH BIOSCIENCE CO., LTD., Taipei (TW)

(21) Appl. No.: 15/621,017

(22) Filed: Jun. 13, 2017

(51) Int. Cl. A61B 5/00; A61B 5/04; A61B 90/00; G06F 17/14

(52) U.S. Cl. CPC A61B 5/0062; A61B 5/04; G01N 21/63; A61B 5/0093; G06F 17/142; A61B 90/36

(57) ABSTRACT

A method for evaluating operational performance of a biological system and apparatus for fetching data required therefor are disclosed. The apparatus includes an emitting unit, an electric potential measuring unit, an analog-to-digital converting unit, and an operating unit. The present invention utilizes excitation of external signal light beams to fetch changed data of electric potential from the skin surface, further to observe the status of the organs inside the living creature's body. In comparison with traditional technologies, such as X-ray and nuclear magnetic resonance, the present invention has advantages of short observation time, low operating cost, and harmless to the living creature.

### FIELD OF THE INVENTION

The present invention relates to a method for evaluating operational performance of a biological system, and an apparatus for fetching data required by the method. More particularly, the present invention relates to a method for evaluating operational performance of all biological systems, such as respiratory system, digestive system and so on, of a living creature by simulating the biological epidermis with light beams to obtain the electronic potential change in the body of the living creature, and an apparatus for fetching necessary data required by the evaluating method.

### BACKGROUND OF THE INVENTION

Generally speaking, in order to know the status of organs or systems in the body of a living creature, further understanding the health situation of the living creature, in addition to being dissected in a medical way, there are many non-destructive methods. Take human being for example. Medical staffs use X-rays to observe the lesion with the rays emitted by the cathode. These rays encounter different components and density of organizations. The penetration rates are not the same. Residues remaining in the imaging film or detector in the last are also not the same. Therefore, images with different brightness can be formed. Another higher resolution imager is Nuclear Magnetic Resonance Imaging (NMRI) apparatus. It uses the principle of nuclear magnetic resonance to analyze the degree of attenuation of energy in different structural environments within an object. With the electromagnetic wave emitted by the applied gradient magnetic field detection, it is able to know the locations and species of the nuclei that make up the organ of this object. Thus, a structural image inside the object can be drawn. Broadly speaking, the above-mentioned techniques, no less than the use of high-density energy to stimulate the human body, and use some physical observations that can be detected from the human body at the same time to speculate or restore the situation within the human body. Sometimes, it is necessary to exert a certain amount of adjuvant to the human body, such as a developer. In addition to the problems such as expensive equipment and some minor injuries caused to the human body, if these techniques can be widely applied to all living creatures, whether it is for academic research or disease treatment, there are a lot of parameters needed to be adjusted. There is still much to be improved.

As we all know, living creatures are made up of a large number of single cells, working together to achieve many of the tasks required for survival. Take human being for example again. The human body is composed of 3.72×10¹³ single cells. These cells accumulate to form tissues, organs and systems. When a cell is biologically stimulated (For example, oxidation, microbial violations, temperature anomalies, and so on), the cell's own genes will adjust the structure and synthesis of various molecular substances, in a certain range, to change abnormal conditions back to normal. Meanwhile, it will also convey its own situation to the neighboring cells, together passing the message to other organs or systems. When these cell biological information and the number of occurrences reach a certain threshold, other organ or system will respond, for example, immune cells began to activate, adrenal gland secretes hormones, pituitary secretes hormones and so on. Its purpose is to assist in the repair. Modern biomedical points out that among cells, organs or systems, for use of molecular materials to convey the messages, in addition to molecular structure and quantity, involved level also includes energy, electrons and so on which are of more subtle changes. In addition to the use of adjacent cells to pass message, other cells, organs and systems may get the relevant information through the nervous system and circulatory system. Hence, in addition to blood, urine and other body fluids test analysis of “relatively giant” metabolic status, to understand the subtle changes of the messages can know more details about the current overall status of various organs and systems in the body. Now, at the molecular biology level, there is a partial understanding of message delivery status in vivo, e. g. energy and lesions of mitochondrion. However, if the skin is properly stimulated and signals, such as voltage changes, from the skin can be read, the current situation of the organ or system that delivers the message may be known.

A related prior art can be found in U.S. Patent Application No. 20160220130. The technology relates to an array physiological detection system which is capable of detecting and recording user's physiological characteristics in three dimensions or more. The invention has the steps of irradiating a light on a skin area such that the light penetrates a surface of the skin area and reaches a dermis of the skin area; continuously detecting an outgoing light that emits outwardly of the dermis of the skin area by each of the plurality of photosensitive pixels to output a brightness variation signal; converting the brightness variation signals of the plurality of photosensitive pixels into a plurality of frequency domain data; calculating a variation value of the plurality of frequency domain data; and identifying a microcirculation state according to a change of the variation value. Although the physiological detection system can stimulate the skin with a light of specific wavelengths and read the skin's response. However, since light beams are in and out like effect a mouse detecting desktop texture, it can only know the changed signal from shallow microcirculation of blood vessels which rise and fall changes. It cannot read the responses to the light beams from other organs or systems in the body. Applications are limited a lot.

Hence, in order to effectively use external stimulation of light to understand the operation of the systems of the human body, even further accessing aging and damage of every system with clinical data, before medical improvement to alert as early as possible, as part of preventive medicine, the inventor provides a method for evaluating operational performance of a biological system and an apparatus to fetch the data required by the method. Importantly, the method can apply not only on human beings, but all living creatures, contributing to scientific researches.

### SUMMARY OF THE INVENTION

This paragraph extracts and compiles some features of the present invention; other features will be disclosed in the follow-up paragraphs. It is intended to cover various modifications and similar arrangements included within the spirit and scope of the appended claims.

In order to fulfill the requirements above, the present invention a method for evaluating operational performance of a biological system. The method comprises the steps of: a) continuously emitting signal light beams with specific wavelengths to a first region of skin of a living creature; b) measuring electric potential of a second region of skin of the living creature at a sampling frequency within a specific time, and converting the measured value of electric potential to a corresponding binary value; c) sequentially fetching a performance data converted from the corresponding binary values which are not within a threshold interval, wherein the performance data contains a plurality of bits of 0 and 1; d) processing fast Fourier transform operation on bits of a plurality of N bytes selected from the performance data; e) fetching M coefficients corresponding to first M number of maximum periodic cosine waves obtained from the fast Fourier transform operation; and f) calculating a scattering relation value based on average values of the M coefficients obtained by executing step a to step e on a plurality of samples of the living creature under normal conditions.

Preferably, the second region and the first region are not on the surface of adjacent skin of the living creature. A way to convert the binary values is calculating an average value of K continuous binary values not within the threshold interval, and converting a central binary value of the K continuous binary values to 1 if it is greater or equal to the average value or to 0 if it is smaller than the average value.

According to the present invention, the specific wavelengths may be near infrared light wavelengths and range from 860 nm to 890 nm. N may be 9, M may be 10 and K may be 21. The electric potential in step b may be measured by an optical diode.

For said method for evaluating operational performance of a biological system, the scattering relation value may be calculated by the steps of: 1) calculating a sum of absolute values of difference amounts between M coefficients and the corresponding average values of the M coefficients and assigning a value from 1 to L according to the calculated sum ranking from small to large, wherein L is a positive integer, the value of 1 represents the difference amount ranging from zero to a next level, and the value of L represents the difference amount ranging from the maximum to the previous level; 2) calculating the amount of values being assigned with 1 to L, respectively; 3) setting L integer values from large to small; and 4) multiplying the number of sets arranged with the value from 1 to L by the corresponding integer value arranged from large to small, respectively, and dividing the sum of the products by a product of the number of sets and the maximum of the integer. L may be 6.

An apparatus for fetching data required by the method for evaluating operational performance of a biological system is also disclosed in the present invention. The apparatus includes: an emitting unit, for continuously emitting signal light beams with specific wavelengths to a first region of skin of a living creature; an electric potential measuring unit, capable of being attached to a second region of the skin of the living creature, for measuring the electric potential of the second region; an analog-to-digital converting unit, electrically connected to the electric potential measuring unit, for converting the measured value of electric potential to a corresponding binary value at a sampling frequency within a specific time; and an operating unit, electrically connected to the analog-to-digital converting unit, for sequentially fetching a performance data containing a plurality of bits of 0 and 1 converted from the corresponding binary values which are not within a threshold interval, processing fast Fourier transform operation on bits of a plurality of N bytes selected from the performance data, fetching M coefficients corresponding to first M number of maximum periodic cosine waves obtained from the fast Fourier transform operation, and calculating a scattering relation value based on average values of the M coefficients obtained by the apparatus for a plurality of samples of the living creature under normal conditions.

Preferably, the second region and the first region are not on the surface of adjacent skin of the living creature. A way to convert the binary values is calculating an average value of K continuous binary values not within the threshold interval, and converting a central binary value of the K continuous binary values to 1 if it is greater or equal to the average value or to 0 if it is smaller than the average value.

According to the present invention, the emitting unit may be an optical diode, and the specific wavelengths are near infrared light wavelengths and range from 860 nm to 890 nm. N may be 9, M may be 10 and K may be 21. The electric potential measuring unit may be an optical diode.

The operating unit further calculates the scattering relation value by the steps of: 1) calculating a sum of absolute values of difference amounts between M coefficients and the corresponding average values of the M coefficients and assigning a value from 1 to L according to the calculated sum ranking from small to large, wherein L is a positive integer, the value of 1 represents the difference amount ranging from zero to a next level, and the value of L represents the difference amount ranging from the maximum to the previous level; 2) calculating the amount of values being assigned with 1 to L, respectively; 3) setting L integer values from large to small; and 4) multiplying the number of sets arranged with the value from 1 to L by the corresponding integer value arranged from large to small, respectively, and dividing the sum of the products by a product of the number of sets and the maximum of the integer. L may be 6.

The present invention utilizes excitation of external signal light beams to fetch changed data of electric potential from the skin surface, further to observe the status of the organs inside the living creature's body. Comparing with the technologies, such as X-ray and nuclear magnetic resonance, the present invention has advantages of short observation time, low operating cost, and harmless to the living creature.

### BRIEF DESCRIPTION OF THE DRAWINGS

FIG. 1 is a flowchart of steps of a method for evaluating operational performance of a biological system according to the present invention.

FIG. 2 illustrates preferable first region and second region.

FIG. 3 describes a relationship between measured electric potentials and converted binary values.

FIG. 4 illustrates human pancreas and duodenum area.

FIG. 5 is a flowchart of steps of calculating a scattering relation value.

FIG. 6 is a schematic diagram of an apparatus for fetching data required by the method for evaluating operational performance of a biological system.

### DETAILED DESCRIPTION OF THE PREFERRED EMBODIMENTS

The following description contains the embodiments of the present invention in order to understand how the present invention is applied to practical conditions. It is to be noted that in the following figures, portions not related to the illustrative techniques of the present invention have been omitted. Meanwhile, In order to highlight the relationship between the elements, the ratio between the components in the diagram and the ratio between the real components is not necessarily the same.

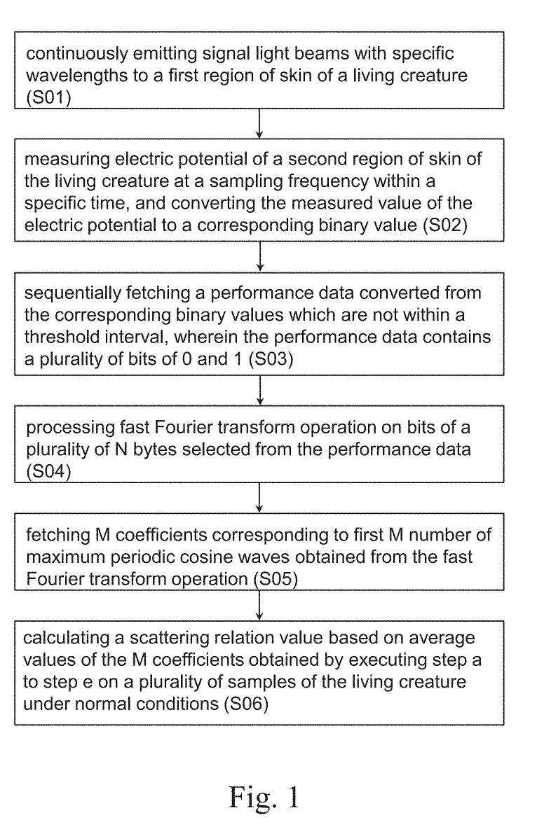

Please refer to FIG. 1. It is a flowchart of steps of a method for evaluating operational performance of a biological system according to the present invention. First, continuously emit signal light beams with specific wavelengths to a first region of skin of a living creature (S01). In this step, the living creature refers to any species that have a living phenomenon, especially an animal. In the present embodiment, take human beings (human body) as an example for illustration. Human beings have many accumulated academic and practical achievements in medicine, and the clinical trials carried out corresponding to the present invention all can be used to verify the specific results of the present invention. The biological system of a living creature mentioned here, for human beings, refer to a physiological system, such as digestive system, reproductive system, endocrine system, etc., which has specific operational objectives and involves at least one organ. General living creatures also have their own physiological systems. Theoretically, the signal light beams emitted to the body through the skin should be better under a situation of no background light, in order to avoid interferences of background light with close wavelengths or strong energy. In practice, the interferences can be eliminated by eliminating background noise. However, in order to avoid unnecessary burst interference or interferences of too strong light sources, resulting in inaccurate evaluation, reduce light intensity near the source of the signal light beams as much as possible.

According to the spirit of the present invention, the signal light beams as an external source of energy to stimulate the body, should be continuously emitted to a first region (the first region will be illustrated with a second region disclosed later) of the skin. According to the test results, it is better to keep the continuous time at least one minute. Because the longer the simulation lasts, the more the response signals corresponding to the simulation can be fetched. It is better to have the duration over one minute. For every living creature, the type of light source used to obtain the best light stimulation for the systems in the body may not be the same. Take human beings as an example. The best light wavelengths of the signal light beams should be near infrared light wavelengths. The specific wavelengths fall between 860 nm and 890 nm. In terms of emission energy, every living creature is also not the same. Considering safety, the energy of the signal light beams exerted to the human body may be under 0.5 Watt, preferably 0.25 Watt. Such strength is sufficient to stimulate enough responses to be measured.

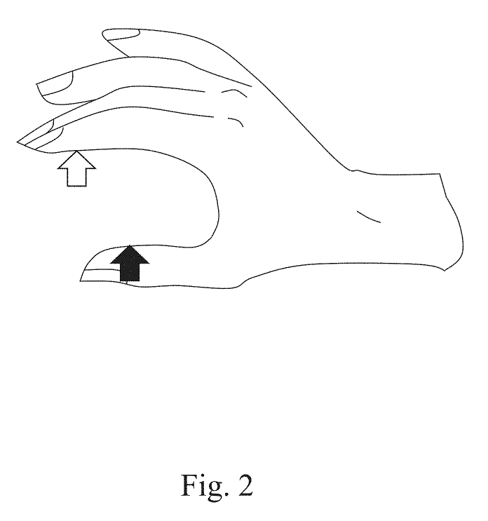

Next, the second step is measuring electric potential of a second region of skin of the living creature at a sampling frequency within a specific time, and converting the measured value of electric potential to a corresponding binary value (S02). In this step, the specific time corresponds to the emitting time of the signal light beams in step S01. During the emitting time of the signal light beams, data for analysis in following steps can be collected. Measuring electric potential can be executed by an optical diode. Sampling frequency may depend on the characteristics of existing electronic components. For example. If an Analog-to-Digital Converter (ADC) with sampling frequency of 100 kHz or 192 kHz and resolution of 12 bits is used, the corresponding sampling frequency is 100 kHz or 192 kHz. About the first region and the second region of the skin, in principle, the second region and the first region are not on the surface of adjacent skin of the living creature. It is to avoid that the source of the signal light beams and the potential measurement side are too close. The received voltage value is locally affected and the observation of the target system is lost. Since it hopes that the simulation of the signal light beams are able to deliver to all parts of the body, emitting of the signal light beams and measuring of the electric potential should be better in the opposite directions. A preferable example is shown in FIG. 2. The signal light beams are emitted along the direction of the hollow arrow to the skin (the first region) opposite to the nail of the index finger. Electric potential is measured on the skin (the second region) on the opposite of the nail of the thumb pointed by the solid arrow.

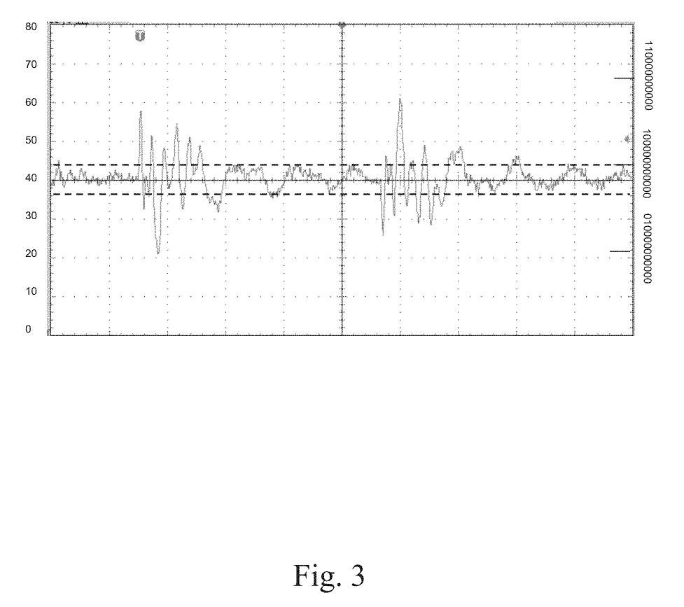

A relationship between measured electric potentials and converted binary values is shown in FIG. 3. It should be emphasized that FIG. 3 is only an example used for illustration in the present embodiment. It may not represent real operation. FIG. 3 shows a plane coordinate system. The horizontal axis represents time. Its unit is second. There are two vertical axes. The unit in mV on the left vertical axis is electric potential of the second region (relative to grounding). The unit on the right vertical axis is binary 12 bits, corresponding to the electric potential values (There are 12 continuous 0 or 1. They represent 0˜4095 if converted to a decimal value. Namely, voltages in a certain range are divided into 4096 values, represented by binary values). Conversion between electric potentials and corresponding binary values is linear conversion. That is, a continuous section of the entity of electric potential values corresponds to a binary value. The binary value corresponding to its adjacent section of electric potential values may be large or less than “1”.

Then, sequentially fetching a performance data converted from the corresponding binary values which are not within a threshold interval, wherein the performance data contains a plurality of bits of 0 and 1 (S03). Please see FIG. 3 again with the following instructions. Since measurement of the electric potential is not continuous. It is to obtain the measured value with a certain frequency in a very short period of time. Real data fluctuate.

As mentioned above, these values may be subject to the interference of background light, affected by the inherent noise of the apparatus when implementing the method. These interferences or noise need to be removed in a simple way to improve the accuracy of the evaluation results. Based on different environments and calibration experiences, it is able to select a threshold interval, using binary values (a value of a 12 bits in the present embodiment), to find out qualified converted binary values. For example, select 011101110010 (dashed line) representing 36 mV and 01111110001 (dashed line) mV as upper and lower limits of the threshold interval. It can be available from FIG. 3 that data within two dashed lines fall in the threshold interval and are not adopted for further analysis. The data not within the two dashed lines can be adopted. It should be emphasized that selection of the threshold interval may be different in every embodiment. It is not limited to what are shown in FIG. 3.

In the present embodiment, a 100k Hz ADC is used. Therefore, 6000k data will be read in one minute for analysis. In fact, after screening of the threshold interval, there are a lot of data deleted so that there are only 1/60 (in this present embodiment) of the binary values left after conversion. Namely, about 98.3% of the binary values are deleted and there are only 100k data left. These 100k data are going to be converted to the one can be analyzed. A way to convert the binary values is calculating an average value of K continuous binary values not within the threshold interval, and converting a central binary value of the K continuous binary values to 1 if it is greater or equal to the average value or to 0 if it is smaller than the average value. For example, take K as 21. It means a qualified binary value representing “0” or “1” depends on the average value calculated from the qualified binary values in front of and in back of it. For example, the average value is 101110010011. If the central binary value is 101100011011, smaller than the average value, then said binary value is converted to. Otherwise, it is 1. Therefore, 100k data become 100k bits of 0 and 1. This is so called performance data. Ideally, 100kbits for the performance data is enough. Performance data with more bits will lead to more accurate evaluation results. However, it will greatly consume computer resources and should be choose carefully. It should be noticed that these bits must be arranged sequentially. Data sampled later is arranged later.

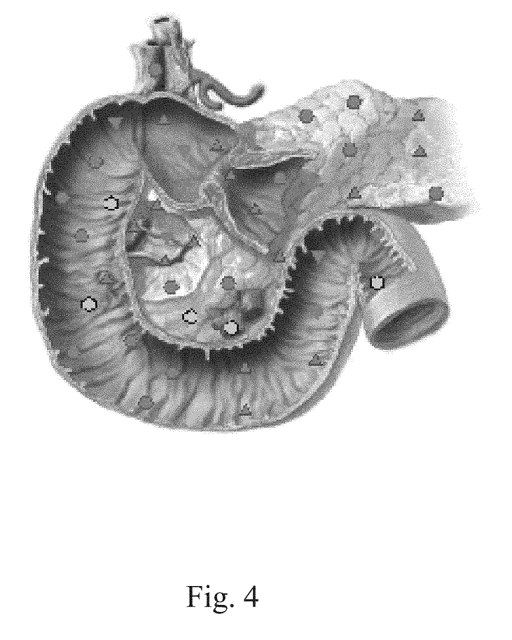

The next step of the method is processing fast Fourier transform operation on bits of a plurality of N bytes selected from the performance data (S04). A theoretical basis of the present invention is when all cells in a certain range work, they will response to the signal light beams. Feedbacks of the response will result in electric potential change on the surface of the skin. Therefore, bits (specific 8N values, 0 or 1) of N bytes of the step represents the responses of the cells in the certain range. After many clinical trials, N is selected as 9. It can optimize the accuracy of the evaluation results and reduce use of computer resources when analyzing. Relationship of correspondence between bits of N bytes and range of cells is found through clinical trials. It should be noticed that, in the experiments, it is also found that feedbacks from cells of some ranges may reflect to the same bits of N bytes. About practical application, please refer to FIG. 4. FIG. 4 illustrates human pancreas and duodenum area. They belong to the digestive system. There are a large number of dots and triangles, which represent the center of a range of cells. Each corresponds to some bits of one set of N bytes in the performance data. Assuming there are 60 points in FIG. 4, there are corresponding bits of 60 sets of N bytes are used for analysis (processing fast Fourier transform operation). 60 sets of N bytes may not adjacent to one another. Selection of the sets of N bytes should go through a lot of tests to find their corresponding locations.

Next, fetching M coefficients corresponding to first M number of maximum periodic cosine waves obtained from the fast Fourier transform operation (S05). After fast Fourier transform operation, the bits of 60 sets of N bytes are expended to Fourier series, f(x)P=a₀ₚ+a₁ₚcos(x)+a₂ₚcos)2x)+a₃ₚcos(3x)+ . . . , P=1˜60. Every Fourier series has infinite items. Each item represents a periodic function in the range from negative infinity to positive infinity. a0p corresponds to an infinite period. Let M=10. 10 coefficients corresponding to cosine waves with top 10 periods in each set are a₀ₚ, a₁ₚ, a₂ₚ, a₃ₚ, a₄ₚ, a₅ₚ, a₆ₚ, a₇ₚ, a₈ₚ, and a₉ₚ. Selected M is the bigger the better. However, larger values represent larger amount of calculation the subsequently. 10 is a better preferable in this embodiment. It can balance accuracy of the results and the consumed computer resources.

The last step of the method is collecting sets of M coefficients from step S05 to calculate a scattering relation value from M coefficients in each set of and average values of corresponding M coefficients obtained by executing step S01 to step S05 for a number of samples of the living creature (human beings) under normal conditions (S06). From the example, it is to know that for pancreas and duodenum of the digestive system, 60 sets with 10 data in each set can be obtained after a cycle from step S01 to step S05. Take a number of samples under normal conditions, e.g. 1000 samples, to repeat those steps. For the digestive system, there are 1000 mentioned data. Calculate average values for the data. There are also 60 sets, 10 average values in one set. This can represent the normal (healthy) condition of the human digestive system. Take 60 sets with 10 average values in one set from a special case to calculate with the average values under normal condition, the scattering relation value can be obtained. The scattering relation value is the final index for the present invention. The closer the index approaches 1, the more normal the operation of the physiological system is.

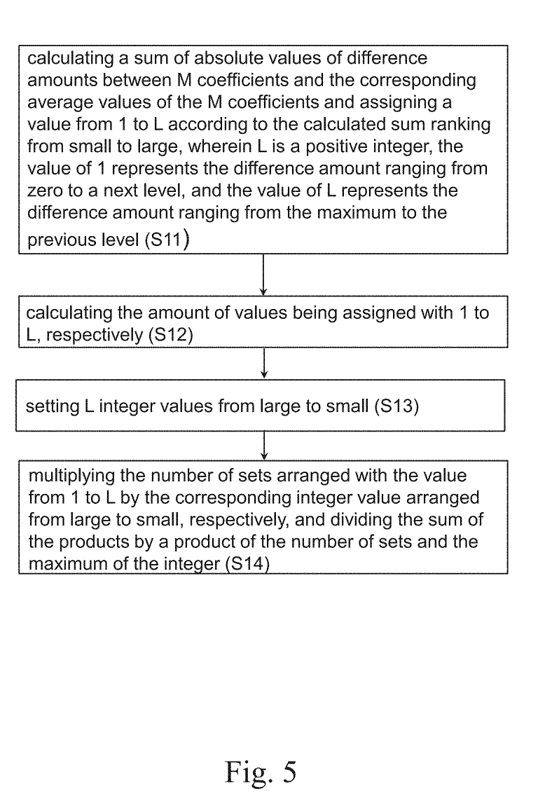

Regarding the way to calculate the scattering relation value, please refer to FIG. 5. It is a flowchart of steps of calculating a scattering relation value. First, calculate a sum of absolute values of difference amounts between M coefficients and the corresponding average values of the M coefficients and assign a value from 1 to L according to the calculated sum ranking from small to large. L is a positive integer. The value of 1 represents the difference amount ranging from zero to a next level, and the value of L represents the difference amount ranging from the maximum to the previous level (S11). Let L=6. It means the value ranges from 1 to 6. If a maximal value of the sum of the absolute values of the difference amounts is 3.6, every 0.6 is a level. Each level represents a specific value, e.g. 0˜0.6 for the value of 1, 0.6˜1.2 for the value of 2, etc. Each level has its assigned value.

Next, calculate the number of sets for the value of 1 to the value of L (S12). For example, there are 5 sets with the value of 1, 10 sets with the value of 2, 20 sets with the value of 3, 15 sets with the value of 4, 10 sets with the value of 5, and no sets with the value of 6. Next step: set L integer values from large to small (S13). For example, 10, 9, 8, 5, 2 and −1. The integer can be negative. Differences between the integers can be any positive integer. Its purpose is to adjust the warning level when there may be something wrong with a system of the body. The more intensive the integers are, the closer the warning level approaches 1. In practice, step S12 and step S13 can be exchanged.

Finally, multiply the number of sets arranged with the value from 1 to L by the corresponding integer value arranged from large to small, respectively, and dividing the sum of the products by a product of the number of sets and the maximum of the integer (S14). According to the example above, it is calculated by (5×10+10×9+20×8+15×5+10×2+0×(−1))/(60×10)=0.658. It there are 60 sets with the value of 1, the result of the calculation is 1. In other words, one observation is the same as the condition the average values indicates. 0.658 shows that the physiological system may have a problem.

The present method doesn't limit to evaluate operational performance of one system of the living creature only. This method can also be applied if a disease affects a particular organ or multiple systems. Set up multiple organs that cross several biological physiological systems (namely, selection of bits of sets of N bytes) and process observation and analysis. The results can show the extent of influence by the disease.

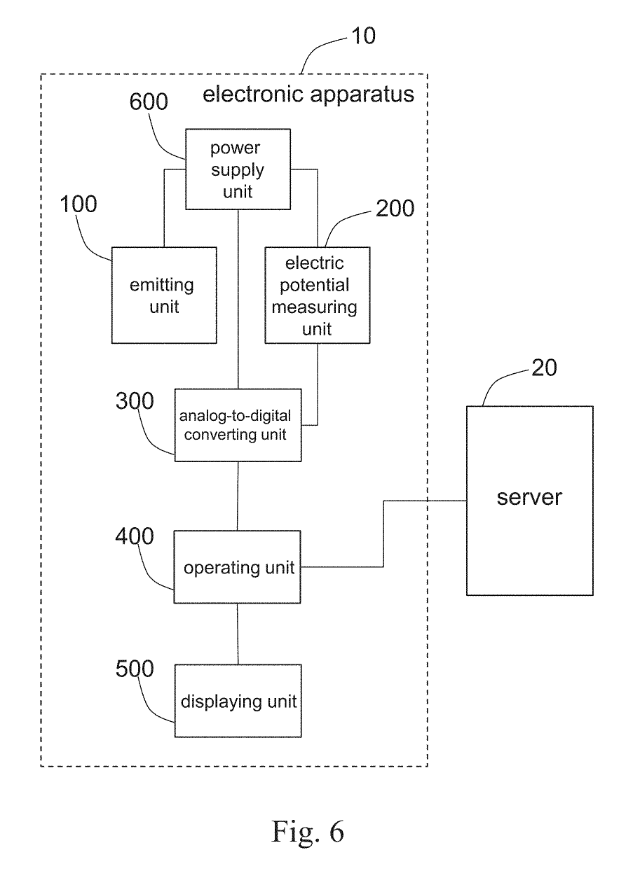

In fact, the method for evaluating operational performance of a biological system provided by the present invention can be implemented by an electronic apparatus 10 to fetch related data. The electronic apparatus 10 is shown in FIG. 6. It includes an emitting unit 100, an electric potential measuring unit 200, an analog-to-digital converting unit 300, an operating unit 400, a displaying unit 500, and a power supply unit 600. Functions of these elements are described below.

The emitting unit 100 can continuously emit signal light beams with specific wavelengths to a first region of skin of a living creature. Preferably, the emitting unit 100 is an optical diode, capable of emitting light beams with near infrared light wavelengths which range from 860 nm to 890 nm. The electric potential measuring unit 200 is able to be attached to a second region of the skin of the living creature, for measuring the electric potential of the second region. It can be an optical diode. Differences between the first region and the second region have been described above. It is not mentioned here. The analog-to-digital converting unit 300 and the electric potential measuring unit 200 are electrically connected. It is used to convert the measured value of the electric potential to a corresponding binary value at a sampling frequency within a specific time.

The operating unit 400 and the analog-to-digital converting unit 300 are electrically connected for sequentially fetching a performance data containing a plurality of bits of 0 and 1 converted from the corresponding binary values which are not within a threshold interval, processing fast Fourier transform operation on bits of a plurality of N bytes selected from the performance data, fetching M coefficients corresponding to first M number of maximum periodic cosine waves obtained from the fast Fourier transform operation, and calculating a scattering relation value based on average values of the M coefficients obtained by the electronic apparatus 10 for a number of samples of the living creature under normal conditions. It is to say that the operating unit 400 can execute all calculating steps for the scattering relation value in FIG. 5.

The displaying unit 500 and the operating unit 400 are electrically connected for displaying the results of data processing from the operating unit 400. The power supply unit 600 is electrically connected to all units mentioned above for providing necessary power when operating.

The electronic apparatus 10 may further connect to a server 20 wiredly or wirelessly. The server 20 has a database. The database can store said M coefficients obtained from the samples under normal conditions by the electronic apparatus 10 and corresponding average values for each system of the living creature. Since the operating unit 400 in the electronic apparatus 10 doesn't have enough storage space while the total coefficients and average values are very large in size, it must rely on the database in the server 20 to assist storage, and the database can update its values at any time.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A method for evaluating operational performance of a biological system, comprising the steps of:
a) continuously emitting signal light beams with specific wavelengths to a first region of skin of a living creature;
b) measuring electric potential of a second region of skin of the living creature at a sampling frequency within a specific time, and converting the measured value of electric potential to a corresponding binary value;
c) sequentially fetching a performance data converted from the corresponding binary values which are not within a threshold interval, wherein the performance data contains a plurality of bits of 0 and 1;
d) processing fast Fourier transform operation on bits of a plurality of N bytes selected from the performance data;
e) fetching M coefficients corresponding to first M number of maximum periodic cosine waves obtained from the fast Fourier transform operation; and
f) calculating a scattering relation value based on average values of the M coefficients obtained by executing step a to step e on a plurality of samples of the living creature under normal conditions.

2. The method for evaluating operational performance of a biological system according to claim 1, wherein the second region and the first region are not on the surface of adjacent skin of the living creature.

3. The method for evaluating operational performance of a biological system according to claim 1, wherein a way to convert the binary values is calculating an average value of K continuous binary values not within the threshold interval, and converting a central binary value of the K continuous binary values to 1 if it is greater or equal to the average value or to 0 if it is smaller than the average value.

4. The method for evaluating operational performance of a biological system according to claim 1, wherein the specific wavelengths are near infrared light wavelengths and range from 860 nm to 890 nm.

5. The method for evaluating operational performance of a biological system according to claim 1, wherein N is 9.

6. The method for evaluating operational performance of a biological system according to claim 1, wherein M is 10.

7. The method for evaluating operational performance of a biological system according to claim 1, wherein K is 21.

8. The method for evaluating operational performance of a biological system according to claim 1, wherein the electric potential in step b is measured by an optical diode.

9. The method for evaluating operational performance of a biological system according to claim 1, wherein the scattering relation value is calculated by the steps of:
1) calculating a sum of absolute values of difference amounts between M coefficients and the corresponding average values of the M coefficients and assigning a value from 1 to L according to the calculated sum ranking from small to large, wherein L is a positive integer, the value of 1 represents the difference amount ranging from zero to a next level, and the value of L represents the difference amount ranging from the maximum to the previous level;
2) calculating the amount of values being assigned with 1 to L, respectively;
3) setting L integer values from large to small; and
4) multiplying the number of sets arranged with the value from 1 to L by the corresponding integer value arranged from large to small, respectively, and dividing the sum of the products by a product of the number of sets and the maximum of the integer.

10. The method for evaluating operational performance of a biological system according to claim 9, wherein L is 6.

11. An apparatus for fetching the data by the method for evaluating operational performance of a biological system according to claim 1, comprising:
an emitting unit, for continuously emitting signal light beams with specific wavelengths to a first region of skin of a living creature;
an electric potential measuring unit, capable of being attached to a second region of the skin of the living creature, for measuring the electric potential of the second region;
an analog-to-digital converting unit, electrically connected to the electric potential measuring unit, for converting the measured value of electric potential to a corresponding binary value at a sampling frequency within a specific time; and
an operating unit, electrically connected to the analog-to-digital converting unit, for sequentially fetching a performance data containing a plurality of bits of 0 and 1 converted from the corresponding binary values which are not within a threshold interval, processing fast Fourier transform operation on bits of a plurality of N bytes selected from the performance data, fetching M coefficients corresponding to first M number of maximum periodic cosine waves obtained from the fast Fourier transform operation, and calculating a scattering relation value based on average values of the M coefficients obtained by the apparatus for a plurality of samples of the living creature under normal conditions.

12. The apparatus according to claim 11, wherein the second region and the first region are not on the surface of adjacent skin of the living creature.

13. The apparatus according to claim 11, wherein a way to convert the binary values is calculating an average value of K continuous binary values not within the threshold interval, and converting a central binary value of the K continuous binary values to 1 if it is greater or equal to the average value or to 0 if it is smaller than the average value.

14. The apparatus according to claim 11, wherein the emitting unit is an optical diode, and the specific wavelengths are near infrared light wavelengths and range from 860 nm to 890 nm.

15. The apparatus according to claim 11, wherein N is 9.

16. The apparatus according to claim 11, wherein M is 10.

17. The apparatus according to claim 11, wherein K is 21.

18. The apparatus according to claim 11, wherein the electric potential measuring unit is an optical diode.

19. The apparatus according to claim 11, wherein the operating unit further calculates the scattering relation value by the steps of:
1) calculating a sum of absolute values of difference amounts between M coefficients and the corresponding average values of the M coefficients and assigning a value from 1 to L according to the calculated sum ranking from small to large, wherein L is a positive integer, the value of 1 represents the difference amount ranging from zero to a next level, and the value of L represents the difference amount ranging from the maximum to the previous level;
2) calculating the amount of values being assigned with 1 to L, respectively;
3) setting L integer values from large to small; and
4) multiplying the number of sets arranged with the value from 1 to L by the corresponding integer value arranged from large to small, respectively, and dividing the sum of the products by a product of the number of sets and the maximum of the integer.

20. The apparatus according to claim 19, wherein L is 6.
